(19) United States
(12) Patent Application Publication
(10) Pub. No.: US 2017/0246549 A1
(43) Pub. Date: Aug. 31, 2017

(54) ORNAMENT DURABLY EMITTING FRAGRANCE

(71) Applicant: TONGFU MANUFACTURING CO., LTD., Nanjing (CN)

(72) Inventor: Ruimin ZHU, Nanjing (CN)

(73) Assignee: TONGFU MANUFACTURING CO., LTD., Nanjing (CN)

(21) Appl. No.: 15/146,205

(22) Filed: May 4, 2016

(30) Foreign Application Priority Data
Feb. 26, 2016 (CN) 201620142699.X

(51) Int. Cl. A63H 33/00; B44C 5/00; A61L 9/12

(52) U.S. Cl. CPC A63H 33/00; A61L 9/12; B44C 5/005

(57) ABSTRACT

An ornament durably emitting a fragrance includes a shell with a sachet arranged therein, the sachet is made of a non-ventilating sealing bag and fragrant particles in the sealing bag; the bottom of the sealing bag is fixed in the shell, an easy-to-tear opening is arranged at the lateral side of the sealing bag, an upper side of the easy-to-tear opening is fixedly connected with a traction line, the other end of the traction line penetrates through a hole at the top of the shell, and the traction line extending out of the hole is fixedly connected with a pull ring. The ornament is internally provided with a sachet emitting the fragrance, in a production, transportation and selling process, the fragrance in the sachet cannot be dissipated, and when using the ornament, a consumer pull a traction line to open the sealed sachet to emit the fragrance in the sachet.

### TECHNICAL FIELD

The present utility model belongs to the technical field of toys, and particularly relates to an ornament durably emitting a fragrance.

### BACKGROUND

An existing toy or sachet capable of emitting a fragrance for a family is longer in consumed time in a production, transportation or selling process, a particle bag or the sachet for the family in the toy always naturally emits the fragrance in the process, since time that the fragrant particles emit the fragrance is limited, when the product gets to a consumer, not much fragrance is left, if the product of the consumer cannot durably emit the fragrance, the product cannot give full play to its original usage, and a selling amount of the product is greatly reduced.

### SUMMARY

The present utility model aims to overcome defects of the prior art, and provides an ornament durably emitting a fragrance, a sachet emitting the fragrance is arranged in the ornament, in a production, transportation and selling process, the sachet is in a non-ventilating sealing state, the fragrance in the sachet cannot be dissipated, and when using the ornament, a consumer pulls a traction line to open the sealed sachet to emit the fragrance in the sachet. After getting to the consumer, the ornament can keep the fragrance for a long time.

The ornament durably emitting a fragrance comprises a shell, wherein a sachet is arranged in the shell, the sachet consists of a non-ventilating sealing bag and a plurality of fragrant particles in the sealing bag; the bottom of the sealing bag is fixed in the shell, an easy-to-tear opening is arranged at the lateral side of the sealing bag, an upper side of the easy-to-tear opening is fixedly connected with a traction line, the other end of the traction line penetrates through a hole at the top of the shell, and one end of the traction line extending out of the hole is fixedly connected with a pull ring.

Since the sachet adopts the non-ventilating sealing bag, the fragrance in the sachet cannot be dissipated, and the arranged easy-to-tear opening is convenient for a user to tear the sachet open when the user pulls the traction line. When the ornament is required to emit the fragrance, the pull ring on the traction line outside the shell is pulled, the traction line is stressed to pull the sealing bag above the easy-to-tear opening, since the bottom of the sealing bag is fixed at the bottom of the shell, in a process of pulling the traction line, the sealing bag is torn open at the easy-to-tear opening, and the fragrant particles are exposed out of the sealing bag and emit the fragrance.

Further, the ornament further comprises a chuck arranged in the shell, wherein the chuck fixedly sleeves the middle of the traction line, and a diameter of the chuck is larger than that of the hole. Since the traction line penetrates through the chuck, the diameter of the chuck is larger than the diameter of the hole, after the traction line is pulled to tear the sealing bag open, the traction line is continuously pulled outwards, the chuck fixed on the traction line moves to the hole, the chuck is clamped at the hole in the shell, the traction line is prevented from being pulled out, and the pull ring can serve as an ornament hanger.

Preferably, the sealing bag is flat. The flat sealing bag is more convenient to fix and tear open.

Further, a sealing side is arranged at the lateral side of the sealing bag, and the easy-to-tear opening is arranged at the sealing side. The sealing bag is arranged at the sealing side, and can prevent the fragrance in the sealing bag from being dissipated from the easy-to-tear opening.

The present utility model has the beneficial effects that the ornament durably emitting a fragrance provided according to the present utility model is internally provided with a sachet emitting the fragrance, in a production, transportation and selling process, the fragrance in the sachet cannot be dissipated, and when using the ornament, a consumer pulls a traction line to open the sealed sachet to emit the fragrance in the sachet. After getting to the consumer, the ornament can keep the fragrance for a long time.

### BRIEF DESCRIPTION OF THE DRAWINGS

FIG. 1 is a sectional view of the present utility model.

FIG. 2 is an external schematic diagram of the present utility model.

FIG. 3 is a schematic diagram of an opened state of a sachet in an ornament.

Marks description of the drawings: 1, shell, 2, traction line, 3, pull ring, 4, chuck, 5, sealing bag, 6, easy-to-tear opening, 7, sealing side, 8, fragrant particles, 9, hole

### DETAILED DESCRIPTION

The present utility model is further described in combination with drawings and a specific embodiment.

### Embodiment

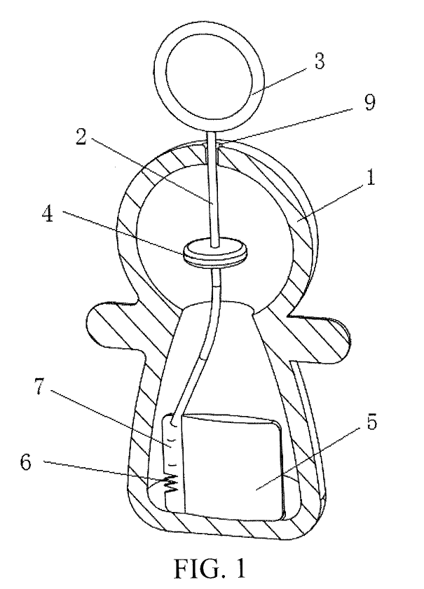

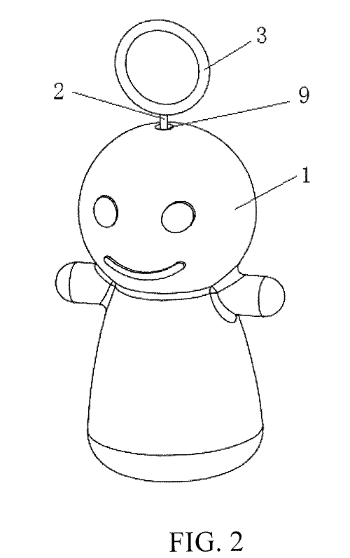

An ornament durably emitting a fragrance as shown in FIG. 1 and FIG. 2 comprises a shell 1, wherein a sachet is arranged in the shell 1, the sachet consists of a non-ventilating sealing bag 5 and a plurality of fragrant particles 8 in the sealing bag 5 and the sealing bag 5 is flat. The bottom of the sealing bag 5 is fixed in the shell 1, an easy-to-tear opening 6 is arranged at the lateral side of the sealing bag 5, further, a sealing side 7 is connected to the lateral side of the sealing bag 5, and the easy-to-tear opening 6 is arranged on the sealing side 7. The sealing bag 5 is arranged on the sealing side 7, so that the fragrance in the sealing bag 5 is prevented from being dissipated from the easy-to-tear opening 6. The upper side of the easy-to-tear opening 6 is fixedly connected with a traction line 2, the other end of the traction line 2 penetrates through a hole 9 at the top of the shell 1, and one end of the traction line 2 extending out of the hole 9 is fixedly connected with a pull ring 3.

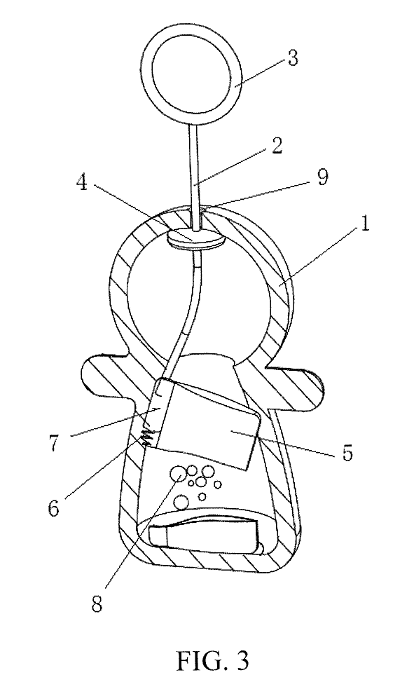

Further, the ornament further comprises a chuck 4 arranged in the shell 1, wherein the chuck 4 fixedly sleeves the middle of the traction line 2, and a diameter of the chuck 4 is larger than that of the hole 9. Since the traction line 2 penetrates through the chuck 4, the diameter of the chuck 4 is larger than the diameter of the hole 9, after the traction line 2 is pulled to tear the sealing bag 5 open, the traction line 2 is continuously pulled outwards, the chuck 4 fixed on the traction line 2 moves to the hole 9, the chuck 4 is clamped at the hole 9 in the shell 1, the traction line 2 is prevented from being pulled out, and as shown in FIG. 3, the pull ring 3 can serve as an ornament hanger.

Since the sachet adopts the non-ventilating sealing bag, the fragrance in the sachet cannot be dissipated, and the easy-to-tear opening 6 is convenient for a user to tear the sachet open when the user pulls the traction line 2. When the ornament is required to emit the fragrance, the pull ring 3 on the traction line 2 outside the shell 1 is pulled, the traction line 2 is stressed to pull the sealing bag 5 above the easy-to-tear opening 6, since the bottom of the sealing bag 5 is fixed at the bottom of the shell 1, in a process of pulling the traction line 2, the sealing bag 5 is torn open at the easy-to-tear opening 6, and the fragrant particles 8 are exposed out of the sealing bag 5 and emit the fragrance.

The ornament durably emitting a fragrance provided according to the present embodiment is internally provided with a sachet emitting the fragrance, in a production, transportation and selling process, the fragrance in the sachet cannot be dissipated, and when using the ornament, a consumer pull a traction line to open the sealed sachet to emit the fragrance in the sachet. After getting to the consumer, the ornament can keep the fragrance for a long time.

The above is merely examples of the present utility model and not limitative to the present utility mode, and all designs same or similar to the present utility model falls within a protective scope of the present utility mode.

1. An ornament capable of emitting a fragrance, comprising a shell wherein: a sachet is arranged in the shell, the sachet consists of a non-ventilating sealing bag and a plurality of fragrant particles in the sealing bag; the bottom of the sealing bag is anchored to the shell, an easy-to-tear opening is arranged at the lateral side of the sealing bag, an upper side of the easy-to-tear opening is fixedly connected with a traction line, the other end of the traction line penetrates through a hole at the top of the shell, and one end of the traction line extending out of the hole is fixedly connected with a pull ring.

2. The ornament capable of emitting a fragrance according to claim 1, further comprising a chuck arranged in the shell, wherein the chuck fixedly sleeves the middle of the traction line, and a diameter of the chuck is larger than that of the hole.

3. The ornament capable of emitting a fragrance according to claim 1, wherein: the sealing bag is flat.

4. The ornament capable of emitting a fragrance according to claim 3, wherein a sealing side is arranged at the lateral side of the sealing bag, and the easy-to-tear opening is arranged at the sealing side.
